(19) United States
(12) Patent Application Publication
(10) Pub. No.: US 2008/0269731 A1
(43) Pub. Date: Oct. 30, 2008

(54) Method and apparatus applying patient-verified prescription of high order aberrations

(76) Inventors: Casimir Andrew Swinger, New York, NY (US); Ming Lai, Webster, NY (US)

Correspondence Address: Casimir Andrew Swinger, 304 East 65th Street, New York, NY, 10065, US

(21) Appl. No.: 12/215,954

(22) Filed: Jul. 1, 2008

Related U.S. Application Data
(63) Continuation-in-part of application No. 10/718,451, filed on Nov. 19, 2003, now Pat. No. 7,407,285.

(51) Int. Cl. A61F 9/008; A61B 18/20; A61B 3/18; B28B 11/08

(52) U.S. Cl. 606/5; 351/205; 351/239; 425/174.4

(57) ABSTRACT

The present invention contemplates an ophthalmic adaptive-optics instrument to obtain patient-verified prescription of low- and high-order aberrations. The present invention further contemplates a new and improved method and apparatus of customized corneal ablation using a patient-verified prescription of low- and high-order aberrations. The patient-verified prescription of high-order aberrations characterizes the aberration correction needed for optimal visual acuity and enables customized corneal ablation to achieve optimal visual acuity of each individual patient.

This application is a continuation-in-part application of U.S. patent application Ser. No. 10/718,451, filed on Nov. 19, 2003, which claims the benefit of U.S. Provisional Application No. 60/418,211, filed on Nov. 20, 2002.

### FIELD OF THE INVENTION

The present invention relates to a method and apparatus for obtaining a patient-verified prescription of high-order aberrations. In particular, the present invention relates to the method and apparatus for obtaining a patient-verified optical prescription that includes high-order aberrations that can subsequently be used for performance of customized corneal ablation in photorefractive surgery.

### BACKGROUND OF THE INVENTION

Topography link and/or wavefront guided custom ablation is a newly developed technology aiming to achieve supernormal visual acuity through photorefractive surgery. In a custom ablation photorefractive surgical procedure, a computer of the surgical system reads in the patient's data from a topography or wavefront device and controls the scan of a surgical laser beam to generate a customized ablation profile on the subject's cornea. It can thus remove corneal irregularities and correct both low- and high-order optical aberrations of the subject's eye.

It is generally expected that the patient's visual acuity, contrast sensitivity and visual function would be significantly improved once the refractive prescription and any irregularity, or high-order aberrations are removed. These irregularities and high-order aberrations of the subject's eye can be measured objectively by a corneal topographer or ophthalmic wavefront instrument. As the diagnostic instrumentation and the refractive surgery procedures themselves have been refined, however, there has been a controversy with respect to the results that have been observed. It has been found clinically that people with excellent visual acuity, such as jet-fighter pilots, may have high-order aberrations not significantly different from normal eyes, while patients who have undergone a reduction in their high-order aberrations may have visual acuity similar to normal eyes.

It is understood that conventional customized ablation is based on objective diagnostic data of the eye. Conventional customized ablation attempts to make the patient's eye into an aberration-free optical system. On the other hand, visual acuity is rather a subjective phenomenon, involving image recognizing and processing by the human brain. The neurologic processes involved in constructing a visual image are expected to vary between individuals. Therefore, an aberration-free eye may not necessarily produce optimal visual acuity and function in a given patient, and it is thereby conceivable that the optimal visual acuity may be attained by an eye not free of aberrations. This has been the clinical experience. It is well known that the lower order aberrations (refractive sphere and cylinder) as determined objectively with instrumentation needs to be refined clinically, using the patient's subjective response, to allow the patient to choose the required optical prescription that satisfies the entire optical system, comprised of the eye and the brain. Any customized refractive surgical procedure that uses objective data without the subjective participation by the patient may thus be less than ideal.

### SUMMARY OF THE INVENTION

The present invention recognizes the limitation with conventional custom ablation in photorefractive surgery and contemplates a new and improved method and apparatus for customized corneal ablation using a patient-verified prescription of low- and high-order aberrations. The present invention further contemplates an ophthalmic adaptive-optics instrument to obtain the patient-verified prescription of low- and high-order aberrations. The subjective patient-verified prescription of high-order aberrations allows characterization of the requisite aberration correction needed for optimal visual acuity and enables customized corneal ablation to achieve optimal visual acuity for each individual patient.

In one embodiment of the present invention, an ophthalmic adaptive-optics instrument is implemented with an observation target, a deformable mirror, a wavefront sensor, processing electronics, and a subjective feedback control. The instrument enables the patient to look at the observation target via the deformable mirror. The wavefront sensor senses the eye's aberrations also via the deformable mirror. The amount of the aberration compensation imposed by the deformable mirror is adjusted and verified by the patient such that optimal visual acuity can be achieved. The instrument measures the total aberrations of the eye and the residual aberrations required for optimal visual acuity. The instrument can then subtract the residual aberration from the total aberration to provide a patient-verified prescription of low- and high-order aberrations for optimal visual acuity.

Accordingly, an objective of the present invention is to provide a new and improved method and apparatus for customized ablation in photorefractive surgery.

Another objective of the present invention is to provide a new and improved method and apparatus for customized ablation based on the patient-verified prescription of low- and high-order aberrations.

A further objective of the present invention is to provide a new and improved method and apparatus for obtaining patient-verified prescriptions of low- and high-order aberrations.

A further objective of the present invention is to provide a new and improved method and apparatus employing a deformable mirror/adaptive optics element to obtain patient-verified prescriptions of low- and high-order aberrations.

The above and other objectives and advantages of the present invention will become more apparent in the following drawings, detailed description, and claims.

### BRIEF DESCRIPTION OF THE DRAWINGS

FIG. 1 shows schematically an ophthalmic adaptive-optics instrument for obtaining a patient-verified prescription of low- and high-order aberrations.

FIG. 2 shows schematically a surgical station for custom corneal ablation using a patient-verified prescription of low- and high-order aberrations, with a UV (ultraviolet) laser.

FIG. 3 shows schematically a lens making station for custom optics using a patient-verified prescription of low- and high-order aberrations.

FIG. 4 shows schematically a second surgical station for custom corneal ablation using a patient-verified prescription of low- and high-order aberrations, with a fs (femtosecond) laser.

FIG. 5 shows schematically a third surgical station for custom corneal ablation using a patient-verified prescription of low- and high-order aberrations, with a fiber-based fs laser.

### DETAILED DESCRIPTION OF THE DRAWINGS

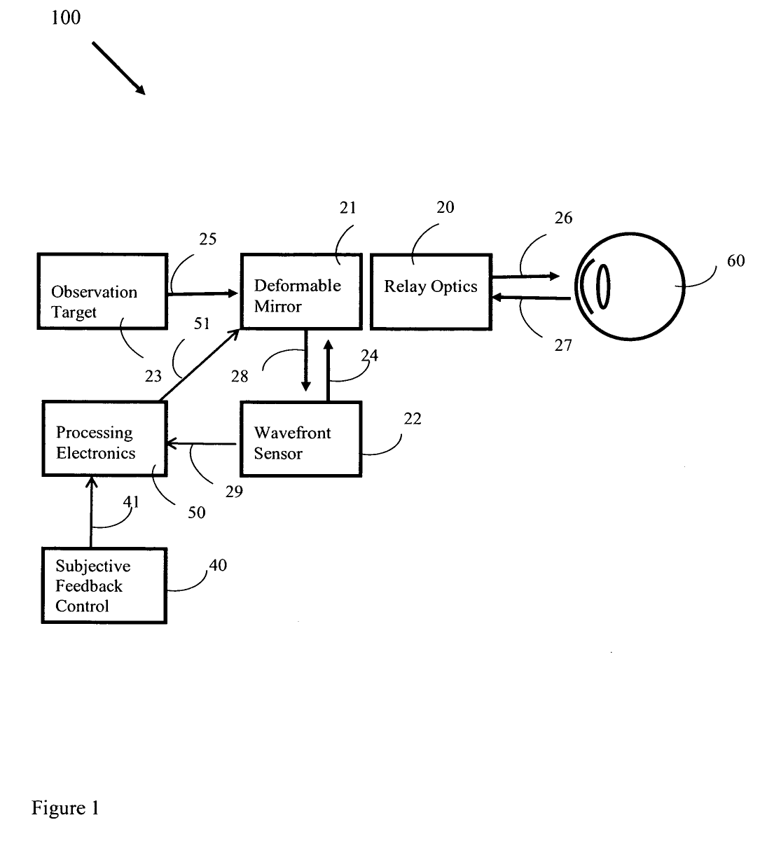

FIG. 1 shows schematically an ophthalmic adaptive-optics instrument 100 for obtaining patient-verified prescriptions of low- and high-order aberrations, in accordance with the present invention. The ophthalmic adaptive-optics instrument 100 consists of relay optics 20, a deformable mirror 21, a wavefront sensor 22, an observation target 23, a subjective feedback control 40, and processing electronics 50.

The relay optics 20 relays the wavefront of an outgoing beam 27 from the pupil plane to the deformable mirror 21. The relay optics 20 comprises two or more lenses with all of their own high-order aberrations well balanced and minimized. The relay optics 20 may include a set of compensation lenses or other mechanism to compensate low-order aberrations of the subject's eye, such as defocus and regular astigmatism. The construction and alignment of relay optics 20 are known to those skilled in the art.

The deformable mirror 21 is used here as an aberration-compensating element to modify or compensate the wavefront distortion of a light beam impinging on it. The deformable mirror 21 is an adaptive-optics element, and it can produce a position-dependent phase modulation across the beam, according to a programmable control signal 51. Therefore, the deformable mirror 21 works as a spatial phase modulator and can be replaced by other types of spatial phase modulators. The construction and control algorithm of a deformable mirror are known to those skilled in the art.

The wavefront sensor 22 projects a probe beam 24/26 into the subject's eye 60 via the deformable mirror 21. The scattered light from the eye's retina forms an outgoing beam 27 from the eye 60. This outgoing beam 27 passes through the deformable mirror 21 and turns into beam 28. The wavefront of the beam 28 is measured with the wavefront sensor 22. The wavefront sensor 22 produces an output signal 29 indicating the aberration of the beam 28. Therefore, the wavefront sensor 22 can measure the total aberration of the eye 60 when the aberration compensation of the deformable mirror 21 is null and the residual aberration of the eye 60 through the deformable mirror 21 with controllable aberration compensation.

The wavefront sensor 22 can be a Hartmann-Shack device or other wavefront sensor such as one operating on the principle of wavefront curvature. The construction and alignment of wavefront sensor 22 are known to those skilled in the art.

The observation target 23 is for the patient to fixate on. It can have an illuminated starburst pattern or other patterns commonly used in ophthalmic instruments. The structure and alignment of observation targets are known to those skilled in the art.

The processing electronics 50 reads in the signal 29 and a command signal 41 and generates a control signal 51 to drive the deformable mirror 21. The deformable mirror 21 thus modifies and compensates the aberration of the subject's eye 60 according to the control signal 51.

In an embodiment of the present invention, the amount of aberration compensation of the deformable mirror 21 is controlled by the command signal 41 from the subjective feedback control 40. This feedback control can be operated by a second party, although the determination of optimal image is always made by the patient. The subjective feedback control 40 is adjusted according to the patient's judgment regarding optimal visual acuity.

In measurement, the patient's eye 60 looks at the observation target 23 through the deformable mirror 21 and the patient makes his or her own judgment regarding optimal visual acuity. First the total aberration of the subject eye 60 is measured and saved as electronic data, while the command signal 41 from the subjective feedback control 40 is set to null. The amount of aberration to be compensated is then adjusted step by step, through the subjective feedback control 40, until an optimal visual acuity is achieved and verified by the patient. If the aberration coma is measured in a subject eye, for example, the compensation for the coma aberration can be made step by step until the subject eye sees an optimal visual acuity. The residual aberration corresponding to the optimal visual acuity is then measured and saved as electronic data.

A patient-verified prescription of low- and high-order aberrations that considers the entire visual system can be then obtained by subtracting the residual aberration from the total aberration of the subject eye 60. The patient-verified prescription carries all the parameters of aberration compensation needed for achieving optimal visual acuity for the subject eye. This patient-verified prescription thus provides the ideal parameters for customized corneal ablation and customized lens making, such as customized contact lenses, customized eyeglasses and customized intraocular lenses.

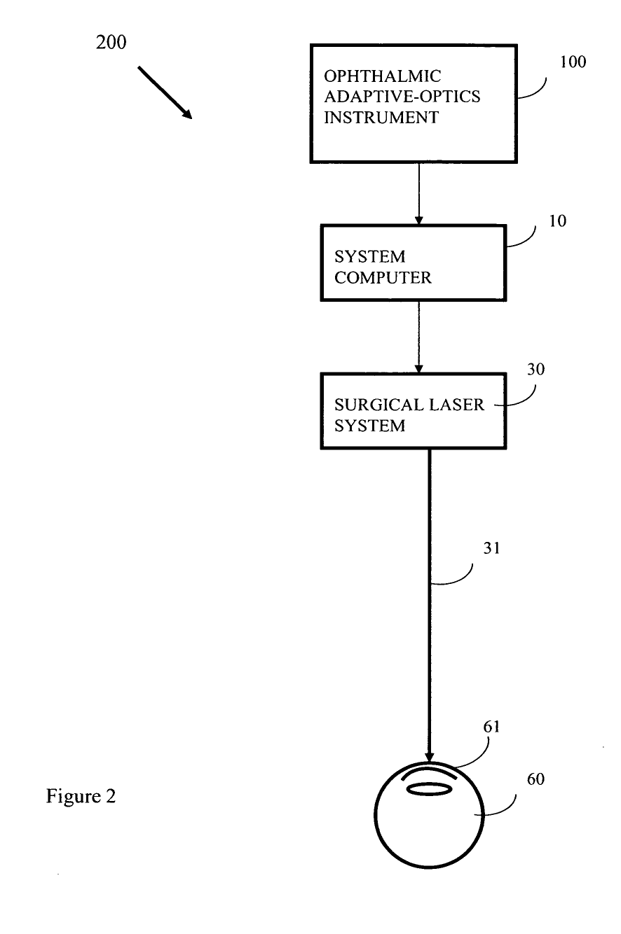

FIG. 2 shows schematically a surgical station 200 for custom corneal ablation using the patient-verified prescription of low- and high-order aberrations with a UV laser. The surgical station 200 consists of an ophthalmic adaptive-optics instrument 100, a system computer 10, and a surgical laser system 30.

The ophthalmic adaptive-optics instrument 100 can be as described in FIG. 1 and is operationally coupled to the system computer 10. The ophthalmic adaptive-optics instrument 100 provides a patient-verified prescription of low- and high-order aberrations and the data of the prescription are saved in electronic format and transmittable through electronic means.

The system computer 10 reads in the patient-verified prescription of low- and high-order aberrations and generates a data file for customized ablation. This data file is to provide an ablation profile that reduces the eye's aberrations to the values where optimal visual acuity shall be achieved according to the patient-verified prescription.

The surgical laser system 30 projects and scans a surgical laser beam 31 onto the cornea 61 of a subject eye 60. The system computer 10 controls the scan of the surgical laser beam 31 to produce a customized ablation profile based on the patient-verified prescription and aimed for optimal visual acuity.

The laser wavelength and fluence of the surgical laser beam 31 are predetermined and are known to those skilled in the art. For the purpose of customized corneal ablation, the surgical laser beam 31 can be delivered from an excimer laser operated at a wavelength of 193 nm and a pulse rate between 10 to 600 Hz. More preferably, the surgical laser beam 31 is delivered from a solid state UV laser source operated at a wavelength around 210 nm and a pulse rate between 200 to 2000 Hz. The surgical laser beam 31 shall have on the cornea a spot size ranging from 300 to 800 microns. To assure accurate ablation, the surgical system shall be equipped with a fast eye-tracking device to compensate the for any eye movement during the surgery. The tracking device is preferably operated at a detection rate of 50 to 5000 Hz.

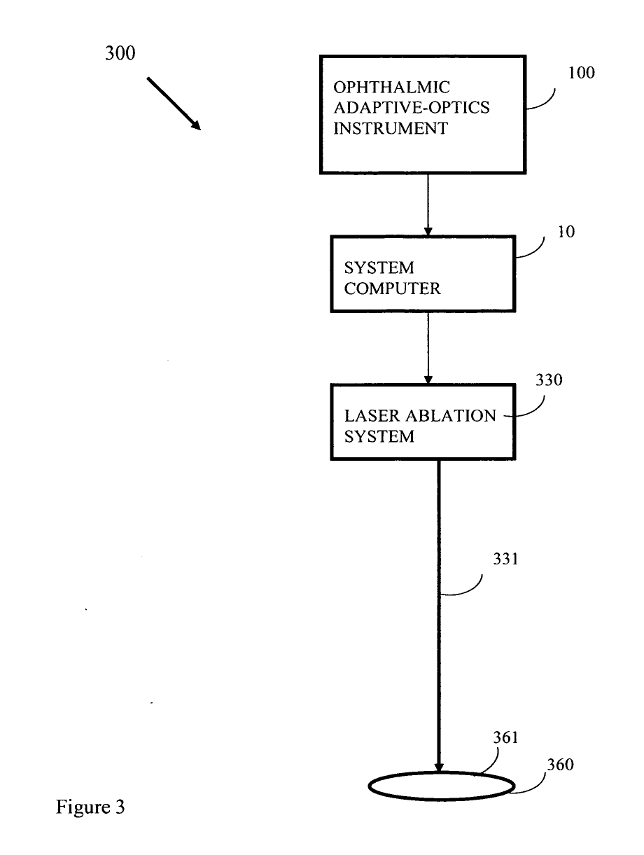

FIG. 3 shows schematically an embodiment of a lens making station 300 for custom optics using a patient-verified prescription of low- and high-order aberrations. The lens making station 300 consists of an ophthalmic adaptive-optics instrument 100, a system computer 10, and a laser ablation system 330. This embodiment of lens making station 300 employs laser ablation to create a custom profile on a surface 361 of a custom lens 360.

The ophthalmic adaptive-optics instrument 100 can be as described in FIG. 1 and is operationally coupled to the system computer 10. The ophthalmic adaptive-optics instrument 100 provides a patient-verified prescription of low- and high-order aberrations and the data of the prescription are saved in electronic format and transmittable through electronic means.

The system computer 10 reads in the patient-verified prescription of low- and high-order aberrations and generates a data file for customized ablation of surface 361 of custom lens 360. This data file is to provide an ablation profile that compensates the eye's aberration to the values where optimal visual acuity shall be achieved according to the patient-verified prescription.

The laser ablation system 330 projects and scans an ablation laser beam 331 onto surface 361 of the custom lens 360. The system computer 10 controls the scan of the ablation laser beam 331 to produce a customized ablation profile based on the patient-verified prescription and aimed for optimal visual acuity.

The laser wavelength and fluence of the ablation laser beam 331 are predetermined and are known to those skilled in the art. For the purpose of custom lens ablation, the ablation laser beam 331 can be delivered from an excimer laser operated at a wavelength of 193 nm if the lens material is PMMA. Other laser wavelengths may be used for custom lens making according to the lens material to be used.

The custom lens 360 can be a contact lens, eyeglasses, or an intraocular lens. The embodiment of lens making station 300 employs laser ablation to create a custom profile on a surface 361 of custom lens 360. Lens making stations employing other mechanisms can also benefit from patient-verified prescriptions to produce custom lenses for optimal visual acuity.

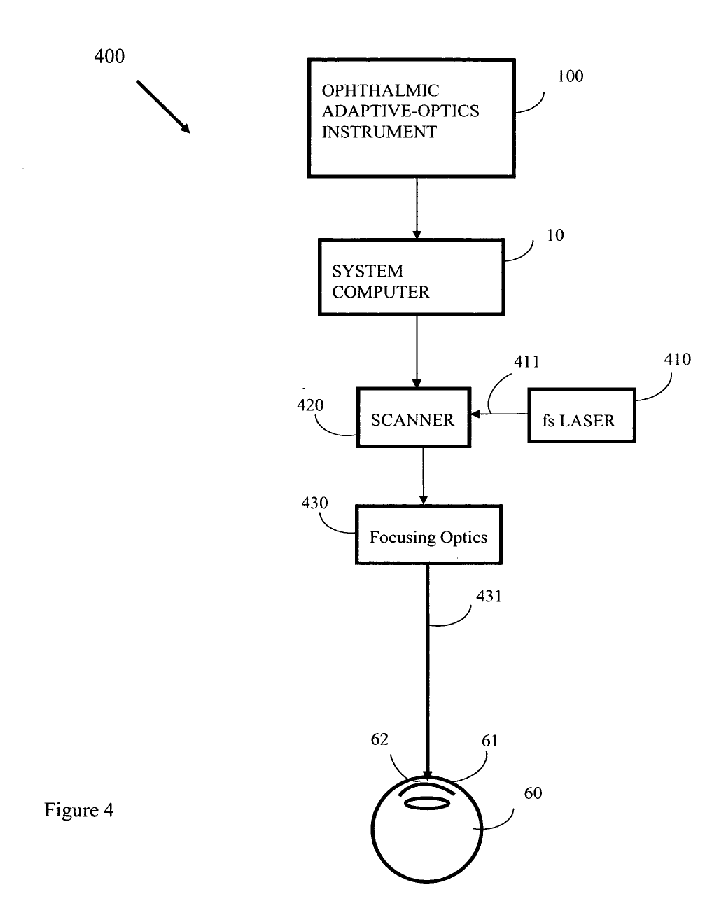

FIG. 4 shows schematically a second surgical station 400 for custom corneal ablation using a patient-verified prescription of low- and high-order aberrations, with a fs laser. The surgical station 400 consists of an ophthalmic adaptive-optics instrument 100, a system computer 10, a fs laser 430, a scanner 420, and a focusing optics 430.

The ophthalmic adaptive-optics instrument 100 performs the same function as described in FIG. 2. The system computer 10 reads in the patient-verified prescription of low- and high-order aberrations and generates a data file for customized ablation inside the corneal stroma 62. This data file is to provide an ablation profile that reduces the eye's aberrations to the values where optimal visual acuity shall be achieved according to the patient-verified prescription.

The fs laser 410 refers to a solid state laser producing femtosecond laser pulses 411 in the near infrared regions of the spectrum (˜1 um). This fs laser 410 is capable to ablate or modify tissue within the depths of the corneal stroma 62 or other intraocular tissue, via intrastromal ablation and cavitation. Swinger et al. describe in U.S. Pat. No. 6,325,792 how fs laser pulses in the near infrared wavelength are used to generate custom ablation profiles inside human cornea. Lubatschowski, et al. describe in US patent publication 2007/0055221 how fs laser pulses with pulse energy of ˜10 nJ and pulse rate up to 50 MHz are used to generate smoother and finer ablation profiles inside human cornea.

The fs laser pulses 411 are directed into the scanner 420 and then to the focusing optics 430. The focusing optics 430 focuses the fs laser pulses 431 inside the corneal stroma 62 of a subject eye 60, while the scanner 420 scans the fs laser beam 431 across the cornea 61. The system computer 10 controls the scan of the fs laser beam 431 to produce a customized ablation profile based on the patient-verified prescription and aimed for optimal visual acuity.

For the purpose of customized intrastromal ablation, the fs laser beam 431 has a wavelength of ˜1 um, a pulse duration between 100-1000 fs, a pulse energy between 5-50 nJ, and a pulse rate between 5-50 MHz. The fs laser beam 431 is focused inside the corneal stroma 62 with a beam waist about 1-5 um. To assure smoother ablation, the scanner 420 may include a fast scanner to produce proper pulse overlap and a slow scanner to generate the calculated ablation profile, as described in US patent publication 2007/0055221 to Lubatschowski, et al.

In another embodiment, this fs laser surgical system 430 is also used to create a corneal flap for a custom PRK or LASIK procedure, as described in U.S. Pat. No. 6,325,792 to Swinger et al. In a further embodiment, the surgical laser system 30 is combined with the fs laser surgical system 430 through a common delivery arm, such that the fs laser system 430 is used to create a corneal flap while the UV laser in the surgical laser system 30 is used to generate the custom ablation profile in accordance with the patient-verified prescription of low- and high-order aberrations. By this way, both the UV surgical laser system 30 and the fs laser surgical system 430 are located on the same side of the patient's bed.

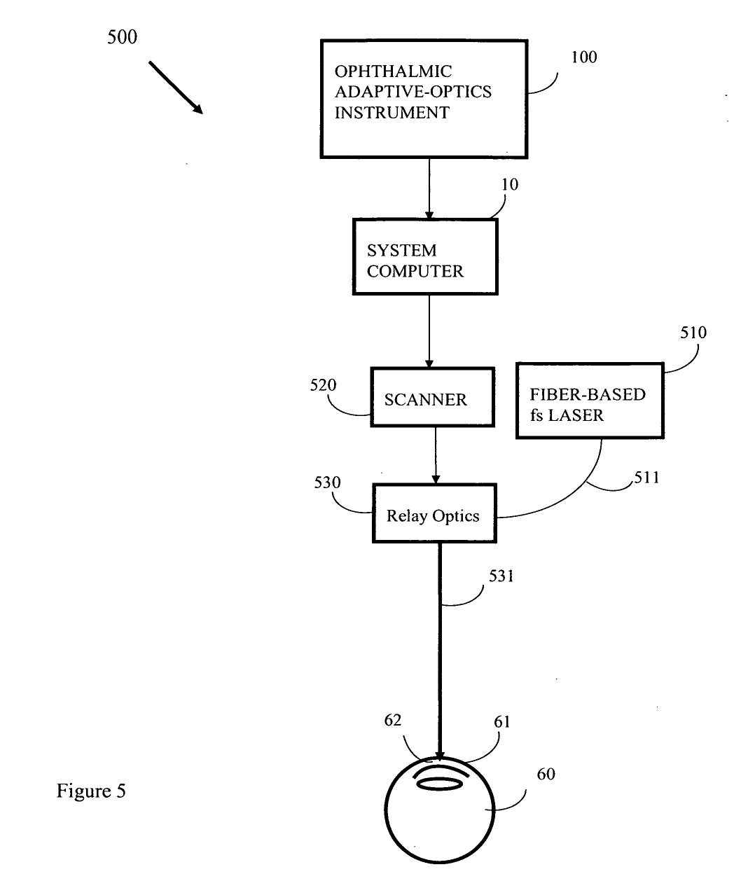

FIG. 5 shows schematically a third surgical station 500 for custom corneal ablation using a patient-verified prescription of low- and high-order aberrations, with a fiber-based fs laser. The surgical station 500 consists of an ophthalmic adaptive-optics instrument 100, a system computer 10, a fiber-based fs laser 510, a pulse-delivering fiber 511, a scanner 520, and relay optics 530.

The ophthalmic adaptive-optics instrument 100 performs the same function as described in FIG. 2. The system computer 10 reads in the patient-verified prescription of low- and high-order aberrations and generates a data file for customized ablation inside the corneal stroma 62. This data file is to provide an ablation profile that reduces the eye's aberrations to the values where optimal visual acuity shall be achieved according to the patient-verified prescription.

The fiber-based fs laser 510 generates fs laser pulses with a wavelength of ˜1 um, pulse duration between 100-1000 fs, pulse energy between 5-50 nJ, and a pulse rate between 5-50 MHz. Such a fiber-based fs laser 510 is commercially available from, for example, PolarOnyx of Sunnyvale, Calif.

The fiber-based fs laser 510 is preferably referred to a diode-pump, fiber laser oscillator, while it may also include a fiber laser oscillator-amplifier system. In a fiber-based fs laser, the laser resonant cavity is fully or substantially confined within optical fiber. Advantageously, a fiber-based fs laser is less sensitive to mechanical vibration, ambient temperature variation and airborne dust. Also advantageously, a fiber-based fs laser can easily implement longer cavity lengths in a more compact configuration in comparison to conventional free-space fs lasers. A longer cavity length is desirable for producing higher pulse energy and lower repetition rate for a given average fs laser power or pump laser power.

The fiber-based fs laser 510 delivers its output beam via an optical fiber 511. The fiber-based fs laser may also include a conventional fs laser oscillator coupled into an optical fiber for fs pulse delivery.

The pulse-delivering fiber 511 delivers fs laser pulses from the fs laser 510 to the relay optics 530. An advantage of the pulse-delivering fiber 511 is the elimination of the need for precise alignment of the laser beam through a long delivery arm, and thus the delivery arm can be made movable or foldable. The pulse-delivering fiber 511 is a single mode fiber, having a core diameter of 5-10 um. The pulse-delivering fiber 511 can also be a polarization-maintained fiber such that the throughput of the fiber is less sensitive to variation in fiber bending and stress.

The scanner 520 and relay optics 530 focuses and scans the fs laser beam 531 inside the corneal stroma 62 of a subject eye 60. The relay optics 530 has a large numerical aperture (e.g., NA=0.2 or larger) to relay a beam waist from the fiber tip of the pulse-delivering fiber 511 into the corneal stroma 62. The system computer 10 controls the scan of the surgical laser beam 431 to produce a customized ablation profile based on the patient-verified prescription and aimed for optimal visual acuity.

Self phase modulation of single mode fiber limits peak power of fs laser pulses delivered. Chirped laser pulses can be used to lower peak power such that higher pulse energy can be delivered through single mode fiber without the onset of significant self phase modulation. In one embodiment, the chirped laser pulses are compressed toward the end of fiber delivery or external to the optical fiber such that optimal pulse duration is achieved inside the corneal stroma or other intraocular element.

Although the above description is based on preferred embodiments, various modifications can be made without departing from the scopes of the appended claims.

### REFERENCES

US PATENT DOCUMENTS

| | | |
|---|---|---|
| 5949521 | Sep. 7, 1999 | Williams et al. |
| 5777719 | | Williams et al. |
| 5144630 | Sep. 1, 1992 | Lin |
| 5520679 | May 28, 1996 | Lin |
| 5782822 | Jul. 21, 1998 | Telfair et al. |
| 5632742 | May 27, 1997 | Frey et al. |
| 5645550 | Jul. 8, 1997 | Hohla |
| 6031854 | Feb. 29, 2000 | Lai |
| 5984916 | Nov. 16, 1999 | Lai |
| 6325792 | Dec. 4, 2001 | Swinger et al. |
| 7101364 | Sep. 5, 2006 | Bille |

US PATENT PUBLICATION

| | | |
|---|---|---|
| US 2002/0013575 | Jan. 31, 2002 | Lai |
| US 2007/0010804 | Jan. 11, 2007 | Rathjen et al. |
| US 2007/0055221 | Mar. 8, 200 | Lubatschowski, |
| | | et al. |

FOREIGN PATENT DOCUMENTS

| | | |
|---|---|---|
| WO 99/55216 | Nov. 4, 1999 | WIPO |
| WO 99/04952 | Feb. 3, 2000 | WIPO |

### OTHER REFERENCES

1. Holger Lubatschowski, Overview of Commercially Available Femtosecond Lasers in Refractive Surgery, Journal of refractive surgery, vol. 24, s102-107, January 2008.

What is claimed is:

1. A laser ablation system applying patient-verified prescription of low- and high-order aberrations, comprising:
an ophthalmic instrument measuring low- and high-order aberrations, wherein said low- and high-order aberrations indicate the amount of aberration correction needed for optimal visual acuity of a subject eye;
adaptive optical means implemented in said ophthalmic instrument to produce aberration correction needed for said optimal visual acuity of said subject eye;
patient-verifying means incorporated in said ophthalmic instrument to provide a patient-verified prescription of low- and high-order aberrations;
a system computer receiving through electronic means said patient-verified prescription and calculating an ablation profile in accordance with said patient-verified prescription; and
a laser ablation system producing an ablation laser beam and having a beam scanning mechanism to scan said laser beam across a vision optics;
wherein said system computer scans said laser beam to produce a customized ablation profile on said vision optics to achieve aberration correction in accordance with said patient-verified prescription.

2. A laser ablation system of claim 1, wherein said laser system is an ophthalmic surgical system and said vision optics is a subject eye.

3. A laser ablation system of claim 1, wherein said laser system is a custom-lens making system and said vision optics is a contact lens, an eyeglass, or an intraocular lens.

4. A laser ablation system of claim 1, wherein said ophthalmic instrument employs a Hartmann-Shack wavefront sensor.

5. A laser ablation system of claim 1, wherein said adaptive optical means includes a deformable mirror.

6. A laser ablation system of claim 1, wherein said patient-verifying means includes an observation target and a subjective feedback control.

7. A laser ablation system of claim 1, wherein said laser ablation system includes an excimer laser operating at a wavelength of 193 nm.

8. A laser ablation system of claim 1, wherein said laser ablation system includes a solid state UV laser operating at a wavelength around 210 nm.

9. A laser ablation system of claim 1, wherein said laser ablation system includes a fs laser operating at a wavelength around 1 um.

10. A laser ablation system of claim 1, wherein said laser ablation system includes a solid-state UV laser having a beam spot size of 300 to 800 um on said vision optics.

11. A laser ablation system of claim 1, wherein said laser ablation system includes a fs laser having a beam waist of 1 to 5 um in said vision optics.

12. A laser ablation system of claim 2, wherein said laser ablation system comprises a fs laser for creating a corneal flap and a UV laser for generating surface ablation.

13. A laser ablation system of claim 12, wherein said fs laser and said UV laser are delivered through a common delivery arm and located on the same side of the patient's bed.

14. A laser ablation system applying patient-verified prescription of low- and high-order aberrations, comprising:
an ophthalmic instrument providing a patient-verified prescription of low- and high-order aberrations, wherein said low- and high-order aberrations indicate the amount of aberration correction needed for optimal visual acuity of a subject eye;
a system computer receiving through electronic means said patient-verified prescription and calculating an ablation profile in accordance with said patient-verified prescription;
a laser system producing fs laser pulses;
a beam scanning mechanism to scan said fs laser pulses across a vision optics; and
focusing optics producing a beam waist of said fs pulses inside said vision optics;
wherein said system computer scans said laser beam to produce a customized ablation profile in said vision optics to achieve aberration correction in accordance with said patient-verified prescription.

15. A laser ablation system of claim 13, further comprising:
an optical fiber to deliver said fs laser pulses from said laser system to said beam scanning mechanism, wherein said optical fiber is a single mode fiber.

16. A laser ablation system correcting low- and high-order aberrations, comprising:
an ophthalmic instrument providing a prescription of low- and high-order aberrations, wherein said low- and high-order aberrations indicate the amount of aberration correction needed for optimal visual acuity of a subject eye;
a system computer receiving through electronic means said prescription and calculating an ablation profile in accordance with said low- and high-order aberrations;
a fiber-based laser system producing fs laser pulses;
a beam scanning mechanism to scan said fs laser pulses across a vision optics; and
relay optics relaying a beam waist of said fs pulses from a fiber output tip to a depth inside said vision optics;
wherein said system computer scans said laser beam to produce a customized ablation profile on said vision optics to achieve aberration correction in accordance with said patient-verified prescription.

17. A laser ablation system of claim 16, further comprising:
an optical fiber to deliver said fs laser pulses from said fiber-based laser system to said beam scanning mechanism, wherein said optical fiber is a single-mode fiber.

18. A laser ablation system of claim 16, further comprising:
an optical fiber to deliver said fs laser pulses from said fiber-based laser system to said beam scanning mechanism, wherein said optical fiber is a polarization-maintained fiber.

19. A laser ablation system of claim 16, further comprising:
chirped pulse means to deliver chirped laser pulses through single mode fiber and to compress said laser pulses to an optimal pulse duration inside said vision optics.

20. A laser ablation system of claim 16, wherein said beam scanning mechanism consists of a fast scanner and a slow scanner.
